(19) United States
(12) Patent Application Publication
(10) Pub. No.: US 2004/0115752 A1
(43) Pub. Date: Jun. 17, 2004

(54) Method for testing samples containing prion protein for the possible presence of the prpsc form

(76) Inventors: Karin Biffiger, Kirchdorf (CH); Markus Moser, Uster (CH); Bruno Oesch, Stilli (CH)

Correspondence Address: RANKIN, HILL, PORTER & CLARK LLP, 4080 ERIE STREET, WILLOUGHBY, OH, 44094-7836, US

(21) Appl. No.: 10/474,107

(22) PCT Filed: Apr. 19, 2002
(86) PCT No.: PCT/EP02/04341

(30) Foreign Application Priority Data
Apr. 21, 2001 (DE) 101 19 713.6

(51) Int. Cl. G01N 33/53; G01N 33/537; G01N 33/543

(52) U.S. Cl. 435/7.93

(57) ABSTRACT

The invention relates to a method for testing samples containing prion protein for the possible presence of the PrP^{Sc} form, according to which: (Step a) the sample is mixed with protease in order to digest protease-sensitive proteins or protein regions; (Step b) after digestion, it is tested whether the sample contains the region PrP 27-30 of the prion protein, which is resistant in the PrP^{Sc} form of the prion protein protease, and the presence of PrP^{Sc} in the sample is established based on the positive detection of PrP 27-30. The inventive method is characterized in that, during Step b, the sample is additionally tested in order to determine whether a complete digestion of the protease-sensitive region of the prion protein has occurred.

### BACKGROUND OF THE INVENTION

The invention relates to a method for testing samples containing prion protein for the presence of a PrP^{Sc} form of prion protein.

Methods for testing samples containing prior proteien for the presence of the PrP^{Sc} form of prion protein currently find their predominant use in the screening of mammals, e.g. animals for slaughtering, for communicable degenerative neurological diseases. Diseases of this type, summarily called spongiform encephalopathies or prion diseases, are known to manifest for instance as BSE in bovines, as scrapie in sheep, or as kuru (laughing disease) or Creutzfeldt-Jakob disease in humans.

As mentioned above, prion diseases are communicable though their infectiousness has not been fully elucidated. The only molecule that has so far been found to be associated with the infectious agent is a disease-specific prion protein (PrP^{Sc}) that constitutes an anomalous isoform of a normal mammalian protein (PrP^{c}) of unknown function. The two isoforms, PrP^{Sc} and PrP^{c}, are identical in terms of their molecular weight and amino acid sequence, but differ in their 3-dimensional folding patterns.

There is much evidence, namely the absence of molecules other than PrP^{Sc} in the prion and especially the absence of nucleic acids, to indicate that. PrP^{Sc} is likely to play the central role in the induction of the diseases mentioned above. PrP^{Sc} proteins are assumed to be capable of converting normal PrP^{c} proteins to the disease-specific folding pattern, which would explain the infectious character of PrP^{Sc} proteins.

Therefore, conventional tests presume PrP^{Sc} to be the central disease-conferring molecule and thus test whether, at least some, of the prion protein contained in a mammalian brain sample, as one example, is present as the PrP^{Sc} form. If this test is positive, then this finding is taken to conclude that the mammal from which the sample was obtained was infected.

As mentioned above, samples from infected sources do not contain PrP_{Sc} exclusively, but also some of the PrP^{c} form of the prion protein. Consequently, the method must provide for differentiation of the PrP^{c} form and any PrP^{Sc} form that may be present.

This issue is being addressed by making use of the fact that the PrP^{c} form can be completely digested with protease whereas only a C-terminal region of the PrP^{Sc} form is protease-sensitive, while a region of the prion protein called PrP 27-30 proves to be resistant to the action of protease.

Therefore, in traditional tests the tested sample is first digested with a protease in a first step (step a) on the assumption that no protease-sensitive regions of the prion protein remain in normal samples and only the protease-resistant region, PrP 27-30, of the PrP^{Sc} form remains in infectious samples after protease digestion. Accordingly, in the second step (step b) of these tests following digestion, it is only tested whether or not the PrP 27-30 region is detectable in the test sample. For detection, these tests use antibodies, as one example, which bind specifically within the PrP 27-30 region. Any antibody-PrP 27-30 complexes thus formed are then detected with common detection methods, e.g. ELISA assays (Moynagh and Schimmel; Nature 1999 Jul 8, 400 (6470): 105). A positive finding in these tests, i.e. the detection of antibody-PrP 27-30 complexes, as one example, is taken as evidence indicating the presence of PrP^{Sc} in the sample which in turn means that the organism from which the sample originated was infected.

One of the shortcomings of the traditional tests has been that they use indirect detection of the agent. In other words: some PrP 27-30 being detectable after digestion is taken as conclusive evidence to indicate that this originated from the protease-resistant region of PrP_{Sc} although the testing method provides no definite differentiation between this region and the corresponding region originating from PrP^{c}. Under unfavorable conditions, e.g. if the sample material is difficult to process, this may lead to false positive results, at least in theory.

### SUMMARY OF THE INVENTION

It is therefore the task of the present invention to further develop methods for testing samples containing prion protein for the presence of a PrP^{Sc} form of prion protein such that they allow a more certain conclusion to be drawn.

The method according to the present invention considers testing the sample in step b after the digestion step (step a) not only for the presence of the region, PrP 27-30, but also to test whether or not the sample still contains protease-sensitive regions of the prion protein.

The method according to the invention thus allows a conclusion to be drawn concerning both the possible presence and absence of PrP 27-30 in the digested sample and whether or not digestion was complete.

### DETAILED DESCRIPTION OF THE PREFERRED EMBODIMENTS

If PrP 27-30 is detected in a digested sample, then this is taken as evidence indicating the presence of PrP^{Sc} only, as long as no protease-sensitive regions of the prion protein are detectable in the digested sample. In contrast, if the sample still contains these protease-sensitive regions after digestion, possible detection of PrP 27-30 is not taken as conclusive evidence indicating the presence of PrP^{Sc}, but may rather mean that the digestion of the corresponding region of the PrP^{c} form may have been incomplete. Under these circumstances, the sample would have to be retested, e.g. at higher protease concentrations or using longer digestion times.

The method according to the invention can therefore be used to exclude false positive results in a particularly certain and simple manner. Especially in the case of rare infectious diseases, such as prion diseases, it is very important for the validity of a test to keep the number of false positive results minimal.

In a preferred embodiment of the invention, PrP 27-30 and protease-sensitive regions of the prion protein are detected by means of molecules that bind specifically within the respective regions of the prion protein, which shall be denoted herein as molecule A (specific for a protease-sensitive region) and molecule B (specific for the PrP 27-30 region).

In a typical method according to this embodiment, the sample would be digested in step a and molecules A and B would be added to the digested sample thereafter, followed by testing whether or not complexes of the prion protein and molecule A and/or molecule B were formed in the sample. The analysis of the results then depends on whether or not complexes were formed and which complexes were formed.

If only complexes of molecule B and prion protein are detected, then the sample does indeed contain PrP^{Sc}. However, if complexes containing molecule A are also present, then there is a risk of obtaining a false positive result. If no complexes or only complexes containing molecule A are detected, then the sample is negative.

Antibodies that specifically recognize the respective regions of the prion protein are particularly well suited for use as molecules A and B (hereinafter referred to as antibodies A and B). However, other molecules showing specific binding, e.g. RNA molecules, can be used equally well for this purpose.

Antibodies recognizing PrP 27-30 have been described and documented in depth, and shall therefore not be further detailed herein.

Antibodies recognizing the protease-sensitive N-terminal region of PrP are known, e.g. from “Brain Research, 545, (1991) 319-321 (Antiserum anti-PrP-N)”, “Brain pathol. 2002; 12; 111 (antibodies FH11, BG4)”, “Proc. Natl. Acad. Sci. Vol. 95 pp. 8812-8815, July 1998 (antibody 5B2)” or “Biochemical and Biophysical Research Communications 273,136-139 (2000) (antibody 8B4)”. The references cited above describe both the properties of the antibodies and their manufacture.

The formation of complexes can be detected by standard methods. Usually, it is considered that one of the two components of the complex formed is bound to a carrier.

Accordingly, it is conceivable, as one example, to immobilize the sample material after digestion, e.g. on a microtiter plate or beads, and then perform the detection with labeled molecules A and B, in particular antibodies A and B. The antibodies, being preferred for this purpose, can be incubated with just one aliquot of the sample material either simultaneously or sequentially. However, it is just as well to prepare two aliquots of the sample in parallel, and then add one or the other of the two antibodies A and B to each sample.

It is also conceivable to immobilize each of the molecules A and B, with these preferably being antibodies A and B, on chips capable of generating a detectable signal in response to a molecular interaction occurring at their surface. Chips of this type are known from EP 887645. Incubation of chips of this type carrying immobilized antibody A or B with the sample material obtained after digestion provides an easy means for measuring, e.g. by optical refraction, whether or not the sample material was bound by the antibodies immobilized on the surface of the chips.

It is preferable to use a sandwich immunoassay for detection. In principle, a sandwich immunoassay of this type utilizes two antibodies per each analyte with these antibodies binding to different epitopes of the analyte. Usually, one of these antibodies is immobilized and serves to couple the analyte to the solid phase, whereas the other antibody is labeled and serves as the detection antibody.

In the present case, the invention considers using another antibody, antibody C, which recognizes PrP 27-30, in addition to antibodies A and B, which recognize the different regions of PrP, wherein antibody C recognizes a different epitope than antibody B.

This presents a number of different options:

It is conceivable to immobilize antibody C on a carrier, incubate the carrier with the sample material obtained after digestion, and then add labeled antibodies A and B for detection.

Another option is to immobilize antibodies A and B on a carrier, incubate the carrier with the sample material, and then add labeled antibody C for detection.

The two latter variants may be associated with some difficulties related to the required signal resolution, standardization, and complications related to the three-fold kinetics.

These difficulties can be resolved by separating the reactions, e.g. by immobilizing the antibodies on different carriers and incubating with separate aliquots of the sample.

A particularly preferred embodiment conceives the use of just one aliquot of the sample such that the sample material obtained after digestion is first incubated with immobilized antibodies A and then with immobilized antibodies B. For detection, labeled antibody C is added as described above. In this embodiment, performing the steps sequentially provides simple means for any protease-sensitive regions of PrP to bind to the specific antibodies A without the kinetics of the binding reaction being affected by the concomitant attack of the antibodies serving as molecules B at the protease-resistant region.

It is conceivable, as one example, to add beads labeled with the respective antibodies to the sample in a sequential fashion or to perform the test with a device, through which the sample material flows and thereby sequentially contacts areas, in which one or the other of the antibodies A or B is immobilized.

As mentioned above, any complexes formed are detected with labeled molecules, in particular with labeled antibodies. If a label is detected or observed on a carrier, then this is taken as evidence indicating that the antibody bearing this label was bound, which, depending on the details of the experimental set-up, may provide evidence of the presence of a certain complex.

Molecules A and B and antibody C may be labeled with the same or different fluorescence markers or enzymes (ELISA) or other suitable markers. In principle, all markers allowing either direct or indirect detection or measurement, are suitable. The various methods of suitably labeling molecules, in particular antibodies, for the methods outlined above and detecting them as part of these methods are known to an expert in this field and are therefore not discussed at any length herein.

1. A method for testing samples containing prion protein for the possible presence of the PrP^{Sc} form, wherein
a) protease is added to the sample in order to digest protease-sensitive proteins or regions of protein,
b) the sample is tested after digestion for the presence of the prion protein region, PrP 27-30, which is protease-resistant in the PrP^{Sc} form of the prion protein, and
c) the detection of PrP 27-30 is taken as conclusive evidence indicating the presence of PrP^{Sc} in the sample,
characterized in that the sample is also tested in step b) for whether or not the protease-sensitive region of the prion protein was digested.

2. A method according to claim 1, characterized in that, in step b), prion protein-binding molecules A and B are added to the sample, wherein molecule A binds within a protease-sensitive region of the PrP protein, and molecule B binds within the PrP 27-30 region, and any complexes of prion protein and molecules A and/or B formed in the sample are detected.

3. A method according to claim 2, characterized in that in that the molecules A and B used in step b) are antibodies.

4. A method according to claim 3, characterized in that the complexes of prion protein and molecules A and/or B formed are detected with a sandwich immunoassay.

5. A method according to claim 4, characterized in that the sample obtained after digestion is first made to contact immobilized antibodies serving as molecules A followed by contacting immobilized antibodies serving as molecules B, and then a labeled antibody recognizing PrP 27-30 is used to detect any complexes of prion protein and immobilized antibodies that may have been formed.

6. A method according to anyone of the claims 2-4, characterized in that the sample is first divided into two aliquots in step b) before one or the other of the molecules A or B is added to each aliquot.
